SEQUENCE LISTING (13 sequences)

SEQ ID NO: 1 - DNA, 2427 bp, Athelia rolfsii
  CDS (1)..(208)
  Intron (209)..(283)
  CDS (284)..(354)
  Intron (355)..(410)
  misc_feature (367)..(367): Nucleotide within intron - any nucleotide.
atgtttcgttcactcctggccttggctgcgtgtgcagtcgcctctgtatctgcacagtctgcgtctgcgacagcatatcttaccaaggaatctgcagttgccaagaatggcgtactttgcaacattggtagccagggatgcatgtctgagggtgcctatagcggtattgtgatcgcatctccctctaaaactagccctgactatctctgtgagtattatttgtaaagtagcctcactgatagtacattttctgagttctgttacaaccctggtattataatagatacctggactcgcgactcgtcgctcgtcttcaagatgttaattgaccaatacacaaatggcctggatacggtatgtggcatcngcgttccggctcgcctcaaagatgnaaaattgatgtttcttagacactgcgcactctcattgacgagtttgtctctgcggaagccaccattcaacaaaccagtaacccatctggtaccgtctctaccggtggtctcggcgaacccaaattcaatatcgacgagacggcatttacgggcgcatggggtcgtgtaagctaccaatacacaatcaaaatcgaccatctgtatttactatctataatttctagccccaacgtgatggtcccgccctccgtgcaaccgcaatcatgacctatgcgacgtatctgtacaacaatggcaacacttcctacgtgaccaacaccctttggcctatcatcaagctcgaccttgactatgtcaactcggactggaaccagaccagtaagcgaatttctagggggacttatctaaaacagcatattcaaccagtaaatagcgtttgacctctgggaagaagttgactcgtcttctttctttacgactgccgttcagcaccgtgctcttgttcagggcgcagcctttgctaccctcatcggccaaacttcgtctgcttcgacttactccgccacggcccctagcattctctgcttcttgcaggtgagataaaaatctttctatgtaattggtttttcccctcaaattgaaattgacatatttgcgatccaatagtcttactggaacaccaacggatactggacggccaacactggtggcggacgttccggcaaggacgccaacaccatactcgcttctatccacacatttgacgccagcgccggctgctctgctgccacgtctcaaccatgctctgacgtagcattggccaacctgaaggtatacgttgactctttccgtagtatttatacgatcaacagcggtatttcctctacctcgggtgttgctactggtcgctaccccgaagattcgtattacaatggcaacgtacgtatttatctaatttttccaagacagtcaaagtttatgttcatctgcccccttttacctgtacattcaaaatagccctggtacctctgcacactcgccgtcgccgagcagctctatgatgctctcatcgtatggaaggctgccggggagctcaacgtcacctccgtctcgctcgcgttcttccagcaattcgactcgagcatcaccgccggcacttacgcctcctcgtcgagcgtatacacttcgctcatctctgacatccaggcgttcgcagacgagtttgttgacattgttgccaagtacacgccttcgtctggcttcttgtctgagcagtatgataagtccacgggtgctcaggattcggctgctaacttgacttgtaagtcatctatttgttcattctattccttttcaaaaaaaaaagtgatgctaatgatttttggcggaaaccagggtcctatgctgctgctatcaccgcttaccaagcccgcaatggcttcacaggtgcttcgtggggtgctaagggagtttctacctcctgctcgactggtgctacaagcccgggtggctcctcgggtagtgtcgaggtcactttcgacgtttacgctaccacagtatatggccgtaagcacttgactagcttcaaaccatacttcatcatgctgataaacaaaaaaatgaaacagagaacatctatatcaccggtgatgtgagtgagctcggcaactggacacccgccaatggtgttgcactctcttctgctaactaccccacctggagtggtaagttgacccttaccagtatcttgacagacattgatattgacttccgcaatacagccacgatcgctctccccgctgacacgacaatccagtacaagtatgtcaacattgacggcagcaccgtcatctgggaggatgctatcagcaatcgcgagatcacgacgcccgccagcggcacatacaccgaaaaagacacttgggatgaatcttaaactgctgaacttgaacggcttgcaaaagcgaatggtgtagaaaataaacgaagattttgattgctttgttttgtttctcttcctatcttgtttctctag

SEQ ID NO: 2 - protein, 579 aa, Athelia rolfsii
MFRSLLALAACAVASVSAQSASATAYLTKESAVAKNGVLCNIGSQGCMSEGAYSGIVIASPSKTSPDYLYTWTRDSSLVFKMLIDQYTNGLDTTLRTLIDEFVSAEATIQQTSNPSGTVSTGGLGEPKFNIDETAFTGAWGRPQRDGPALRATAIMTYATYLYNNGNTSYVTNTLWPIIKLDLDYVNSDWNQTTFDLWEEVDSSSFFTTAVQHRALVQGAAFATLIGQTSSASTYSATAPSILCFLQSYWNTNGYWTANTGGGRSGKDANTILASIHTFDASAGCSAATSQPCSDVALANLKVYVDSFRSIYTINSGISSTSGVATGRYPEDSYYNGNPWYLCTLAVAEQLYDALIVWKAAGELNVTSVSLAFFQQFDSSITAGTYASSSSVYTSLISDIQAFADEFVDIVAKYTPSSGFLSEQYDKSTGAQDSAANLTWSYAAAITAYQARNGFTGASWGAKGVSTSCSTGATSPGGSSGSVEVTFDVYATTVYGQNIYITGDVSELGNWTPANGVALSSANYPTWSATIALPADTTIQYKYVNIDGSTVIWEDAISNREITTPASGTYTEKDTWDES

SEQ ID NO: 3 - DNA, 34 bp, Artificial sequence
  Primer DCrF1
acgtacggatccacaatgtttcgttcactcctgg

SEQ ID NO: 4 - DNA, 30 bp, Artificial sequence
  Primer DCrR1
gtacgtgtcgacctagagaaacaagatagg

SEQ ID NO: 5 - DNA, 40 bp, Artificial sequence
  Primer amp 3′
caaagagacatgggcggccgcaggatcttcacctagatcc

SEQ ID NO: 6 - DNA, 40 bp, Artificial sequence
  Primer Amp 5′
cgtaaccttcatcgcggccgcatgtatccgctcatgagac

SEQ ID NO: 7 - DNA, 40 bp, Artificial sequence
  Primer Ura 3 5′ not
gtctcatgagcggatacatgcggccgcgatgaaggttacg

SEQ ID NO: 8 - DNA, 40 bp, Artificial sequence
  Primer URA 5 3′ not
ggatctaggtgaagatcctgcggccgcccatgtctctttg

SEQ ID NO: 9 - DNA, 19 bp, Artificial sequence
  Primer URA amp 3′
taatcggtaagcgagttgc

SEQ ID NO: 10 - DNA, 19 bp, Artificial sequence
  Primer URA amp 5′
ttacgaatgcacacggtgt

SEQ ID NO: 11 - DNA, 18 bp, Artificial sequence
  Primer DAuP105
gctggtgattggctggct

SEQ ID NO: 12 - DNA, 18 bp, Artificial sequence
  Primer DAuP106
atgttgaatagctcgccc

SEQ ID NO: 13 - DNA, 37 bp, Artificial sequence
  Primer 240303P1
gggcccccgcggctaggggagagcgatcgtggcactc

(19) United States
(12) Patent Application Publication
(10) Pub. No.: US 2006/0270007 A1
(43) Pub. Date: Nov. 30, 2006

(54) Method for producing glucoamylases and their uses

(76) Inventors: Anders Visko Nielsen, Slangerup (DK); Barrie Edmund Norman, Birkerod (DK); Sara Landvik, Vedbaek (DK)

Correspondence Address: NOVOZYMES NORTH AMERICA, INC., 500 FIFTH AVENUE, SUITE 1600, NEW YORK, NY, 10110, US

(21) Appl. No.: 10/578,992

(22) PCT Filed: Jun. 11, 2004
(86) PCT No.: PCT/DK04/00406

§ 371 Date: May 11, 2006

(30) Foreign Application Priority Data
Jun. 13, 2003 (DK) PA 2003 00884

(51) Int. Cl. C12P 19/04; C12P 21/06; C07H 21/04; C12P 7/06; C12N 9/32; C12N 15/74; C12N 1/16

(52) U.S. Cl. 435/101; 435/161; 435/69.1; 435/204; 435/254.3; 435/484; 536/23.2

(57) ABSTRACT

The present invention relates to methods for producing isolated polypeptides having glucoamylase activity, filamentous fungal host cells comprising nucleic acid sequences encoding the polypeptides, and using the polypeptides.

### FIELD OF THE INVENTION

The present invention relates to methods for producing isolated polypeptides having glucoamylase activity, filamentous fungal host cells comprising nucleic acid sequences encoding the polypeptides, and using the polypeptides.

### BACKGROUND

High dextrose and fructose syrups are made by enzymatic saccharification of liquefied starch. The saccharification is achieved by means of a dextrose-forming exo-amylase known as exo-1,4-alpha-D-glucosidase (glucoamylase or amylogulcosidase). The enzyme hydrolyzes 1,4- as well as 1,6-alpha-linkages in starch. During hydrolysis, glucose units are removed in a step-wise manner from the non-reducing ends of the substrate molecule, whereas the 1,6-alpha-linkages found in branched dextrins are broken down relatively slowly. Maltotriose and maltose are hydrolyzed at a lower rate than higher oligosaccharides. Glucoamylases are also used to lower the carbohydrate content of beer.

A method for direct saccharification of raw non-liquefied starch using enzyme, in particular amylase, produced by a basidiomycete strain of Athelia rolfsii (previously denoted Corticium rolfsii) was described in U.S. Pat. No. 4,727,026. A glucoamylase from the same Athelia rolfsii (C. rolfsii) strain was isolated and partially characterized (Nagasaka,Y. et al. (1998) Purification and properties of the raw-starch-degrading glucoamylases from Corticium rolfsii, Appl Microbiol Biotechnol 50:323-330). The gene encoding the G2 form of this glucoamylase was cloned and expressed in a bakers yeast host cell, but the glucoamylase G2 could only be produced in very poor yields (Nagasaka, Y. et al. (1995) Cloning of Corticium rolfsii glucoamylase cDNA and its expression in Saccharomyces cerevisiae, Appl Microbiol Biotechnol 44:451-458).

There is a need in the art for new sources of glucoamylase with improved properties.

### SUMMARY OF THE INVENTION

A problem to be solved by the present invention was how to provide a recombinantly produced glucoamylase of interest in commercially relevant yields. The present inventors have surprisingly found that the glucoamylase of the invention could be expressed in a filamentous fungal host cell, resulting in very high yields of the glucoamylase. They also showed, that the use of this glucoamylase when applied in the treatment of liquefied starch may result in a heretofore unseen high dextrose equivalent (or % dextrose value), thus providing an improved and more economical process for the saccharification of liquefied starch.

Accordingly, in a first aspect the invention relates to a method of recombinantly producing a glucoamylase, said method comprising the step of expressing a polynucleotide encoding a polypeptide having glucoamylase activity (E.C. 3.2.1.3) in a filamentous fungal host cell, wherein the polypeptide comprises an amino acid sequence which is at least 70% identical, preferably at least 75%, or at least 80%, or at least 85%, or 90%, or at least 95%, or even at least 98% identical, to the sequence shown in positions 19 to 471 (both incl.) of SEQ ID NO: 2.

A glucoamylase of Athelia rolfsii, the amino acid sequence of which is available as SPTREMBL:Q12596, is almost identical to the one shown in SEQ ID NO: 2, except for one amino acid residue in position 115 of SEQ ID NO: 2, which in the database sequence is a Serine, whereas in SEQ ID NO: 2 it is a Proline. The annotation of the database sequence identifies amino acid residues 1-18 as a signal peptide, and residues 19-579 as the mature glucoamylase enzyme, with residues 472-482 serving as a linker between the glucoamylase domain and the starch-binding domain comprised in residues 483-579.

The invention also relates to a method of recombinantly producing a glucoamylase, said method comprising the step of expressing a polynucleotide encoding a polypeptide having glucoamylase activity (E.C. 3.2.1.3) in a filamentous fungal host cell, wherein the polypeptide is selected from the group consisting of: (a) a polypeptide encoded by a polynucleotide which hybridizes under high stringency conditions with (i) the polynucleotide shown in positions 1 to 2325 of SEQ ID NO:1, (ii) the cDNA or intron-free polynucleotide contained between nucleotides 1 to 2325 of SEQ ID NO:1, (iii) a subsequence of (i) or (ii) of at least 100 nucleotides, or (iv) a complementary strand of (i), (ii), or (iii); (b) a variant of a polypeptide having an amino acid sequence of SEQ ID NO:2, said variant comprising a substitution, deletion, and/or insertion of one or more amino acids; and (c) a fragment of (a) or (b) that has glucoamylase activity.

In a second aspect, the invention relates to a method for saccharifying liquefied starch, comprising the treatment of the liquefied starch with a polypeptide having glucoamylase activity (E.C. 3.2.1.3), wherein the polypeptide comprises an amino acid sequence which is at least 70% identical, preferably at least 75%, or at least 80%, or at least 85%, or 90%, or at least 95%, or even at least 98% identical, to the sequence shown in positions 19 to 471 (both incl.) of SEQ ID NO: 2, whereby a % dextrose (DX) value of at least 96% is achieved at 30% w/w (g/100 g dry matter) substrate concentration at 60° C., the DX value is determined as defined in Example 7 herein.

The invention also relates to a method for saccharifying liquefied starch, comprising the treatment of the liquefied starch with a polypeptide having glucoamylase activity (E.C. 3.2.1.3), wherein the polypeptide is selected from the group consisting of: (a) a polypeptide encoded by a polynucleotide which hybridizes under high stringency conditions with (i) the polynucleotide shown in positions 1 to 2325 of SEQ ID NO:1, (ii) the cDNA or intron-free polynucleotide contained between nucleotides 1 to 2325 of SEQ ID NO:1, (iii) a subsequence of (i) or (ii) of at least 100 nucleotides, or (iv) a complementary strand of (i), (ii), or (iii); (b) a variant of a polypeptide having an amino acid sequence of SEQ ID NO:2, said variant comprising a substitution, deletion, and/or insertion of one or more amino acids; and (c) a fragment of (a) or (b) that has glucoamylase activity; whereby a % dextrose (DX) value of at least 96% is achieved at 30% w/w (g/100 g dry matter) substrate concentration at 60° C., the DX value is determined as defined in Example 7 herein.

In a third aspect, the invention relates to a filamentous fungal host cell comprising at least one copy of a polynucleotide encoding a polypeptide having glucoamylase activity (E.C. 3.2.1.3), said polypeptide comprising an amino acid sequence which is at least 70% identical, preferably at least 75%, or at least 80%, or at least 85%, or 90%, or at least 95%, or even at least 98% identical, to the sequence shown in positions 19 to 471 (both incl.) of SEQ ID NO: 2.

The invention also relates to a filamentous fungal host cell comprising at least one copy of a polynucleotide encoding a polypeptide having glucoamylase actiity (E.C. 3.2.1.3), wherein the polypeptide is selected from the group consisting of: (a) a polypeptide encoded by a polynucleotide which hybridizes under high stringency conditions with (i) the polynucleotide shown in positions 1 to 2325 of SEQ ID NO:1, (ii) the cDNA or intron-free polynucleotide contained between nucleotides 1 to 2325 of SEQ ID NO:1, (iii) a subsequence of (i) or (ii) of at least 100 nucleotides, or (iv) a complementary strand of (i), (ii), or (iii); (b) a variant of a polypeptide having an amino acid sequence of SEQ ID NO:2, said variant comprising a substitution, deletion, and/or insertion of one or more amino acids; and (c) a fragment of (a) or (b) that has glucoamylase activity.

In a forth aspect, the invention relates to the use of a polypeptide having glucoamylase activity (E.C. 3.2.1.3) in a starch conversion process, in a continuous starch conversion process, in a process for producing oligosaccharides, in a process for producing ethanol for fuel or drinking ethanol (portable alcohol), in a process for producing a beverage, in a fermentation process for producing organic compounds, such as citric acid, ascorbic acid, lysine, glutamic acid, or in detergents, in a process for producing specialty syrups, in the pre-treatment of starch for extrusion, in compost and biological waste treatment, in the purification of plant extracts for food additives, in cosmetics and pharmaceuticals, in the baking industry, in the production of pet food, wherein the polypeptide comprises an amino acid sequence which is at least 70% identical, preferably at least 75%, or at least 80%, or at least 85%, or 90%, or at least 95%, or even at least 98% identical, to the sequence shown in positions 19 to 471 (both incl.) of SEQ ID NO: 2.

### DEFINITIONS

The term “glucoamylase activity” is defined herein as a glucan 1,4-alpha-glucosidase which hydrolyses the terminal 1,4-linked alpha-D-glucose residues successively from non-reducing ends of the chains with release of beta-D-glucose belonging to the Enzyme Class EC 3.2.1.3. For purposes of the present invention, glucoamylase activity is determined according to the procedure described by Fagershom and Kalkkinen, 1995, Biotechnol. Appl. Biochem. 21: 223-231, where the glucose produced by the glucoamylase from 0.1 M maltotriose is measured using a GO glucose oxidase assay kit (Sigma Chemical Co., St. Louis, Mo.) at pH 4, 25° C. One unit of glucoamylase activity is defined as 1.0 μmole of glucose produced per minute at 25° C., pH 4.

### DETAILED DESCRIPTION OF THE INVENTION

In a first embodiment, the present invention relates to a method of recombinantly producing a glucoamylase, said method comprising the step of expressing a polynucleotide encoding a polypeptide having glucoamylase activity (E.C. 3.2.1.3) in a filamentous fungal host cell, wherein the polypeptide comprises an amino acid sequence which is at least 70% identical, preferably at least 75%, or at least 80%, or at least 85%, or 90%, or at least 95%, or even at least 98% identical, to the sequence shown in positions 19 to 471 (both incl.) of SEQ ID NO: 2 (i.e., the mature polypeptide), at least about 75%, preferably at least about 80%, more preferably at least about 85%, even more preferably at least about 90%, most preferably at least about 95%, and even most preferably at least about 97% identical to the sequence shown in positions 19 to 471 (both incl.) of SEQ ID NO: 2. The polypeptides are hereinafter termed “homologous polypeptides”. In a preferred embodiment, the homologous polypeptides have an amino acid sequence which differs by five amino acids, preferably by four amino acids, more preferably by three amino acids, even more preferably by two amino acids, and most preferably by one amino acid from amino acids 19 to 471 of SEQ ID NO:2. For purposes of the present invention, the degree of identity between two amino acid sequences is determined by the ClustaIW method (Higgins, 1989, CABIOS 5: 151-153; Thompson et al., 1994, Nucleic Acids Research 22: 4673-4680; Thompson et al., 1997, Nucleic Acids Research 25: 4876-4882) using the LASERGENE™ MEGALIGN™ software (DNASTAR, Inc., Madison, Wis.) with a blosum weight matrix and gap extension penalty of 0.03 to 0.05 and the following multiple alignment parameters: Gap penalty of 10 and gap length penalty of 10. Pairwise alignment parameters were Ktuple=1, gap penalty=3, windows=5, and diagonals=5.

Preferably, the polypeptides of the present invention comprise the amino acid sequence of SEQ ID NO:2 or an allelic variant thereof; or a fragment thereof that has glucoamylase activity. In a more preferred embodiment, the polypeptide of the present invention comprises the amino acid sequence of SEQ ID NO:2. In another preferred embodiment, the polypeptide of the present invention comprises amino acids 19 to 471 of SEQ ID NO:2, or an allelic variant thereof; or a fragment thereof that has glucoamylase activity. In another preferred embodiment, the polypeptide consists of amino acids 19 to 471 of SEQ ID NO:2 or an allelic variant thereof; or a fragment thereof that has glucoamylase activity. In another preferred embodiment, the polypeptide of the present invention consists of the amino acid sequence of SEQ ID NO:2 or an allelic variant thereof; or a fragment thereof that has glucoamylase activity.

A “fragment” of SEQ ID NO:2 is a polypeptide having one or more amino acids deleted from the amino and/or carboxyl terminus of this amino acid sequence. Preferably, a fragment contains at least 400 amino acid residues, more preferably at least 440 amino acid residues, and most preferably at least 450 amino acid residues.

An “allelic variant” denotes any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

In another embodiment, the present invention relates to a method of recombinantly producing a glucoamylase, said method comprising the step of expressing a polynucleotide encoding a polypeptide having glucoamylase activity (E.C. 3.2.1.3) in a filamentous fungal host cell, wherein the polypeptide is selected from the group consisting of: (a) a polypeptide encoded by a polynucleotide which hybridizes under very low stringency conditions, preferably low stringency conditions, more preferably medium stringency conditions, more preferably medium-high stringency conditions, even more preferably high stringency conditions, and most preferably very high stringency conditions with (i) the polynucleotide shown in positins 1 to 2325 of SEQ ID NO:1, (ii) the cDNA or intron-free polynucleotide contained between nucleotides 1 to 2325 of SEQ ID NO:1, (iii) a subsequence of (i) or (ii) of at least 100 nucleotides, or (iv) a complementary strand of (i), (ii), or (iii); (b) a variant of a polypeptide having an amino acid sequence of SEQ ID NO:2, said variant comprising a substitution, deletion, and/or insertion of one or more amino acids; and (c) a fragment of (a) or (b) that has glucoamylase activity (J. Sambrook, E. F. Fritsch, and T. Maniatus, 1989, Molecular Cloning, A Laboratory Manual, 2d edition, Cold Spring Harbor, N.Y.). The subsequence of SEQ ID NO:1 may be at least 100 nucleotides or preferably at least 200 nucleotides. Moreover, the subsequence may encode a polypeptide fragment which has glucoamylase activity. The polypeptides may also be allelic variants or fragments of the polypeptides that have glucoamylase activity.

The nucleic acid sequence of SEQ ID NO:1 or a subsequence thereof, as well as the amino acid sequence of SEQ ID NO:2 or a fragment thereof, may be used to design a nucleic acid probe to identify and clone DNA encoding polypeptides having glucoamylase activity from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 15, preferably at least 25, and more preferably at least 35 nucleotides in length. Longer probes can also be used. Both DNA and RNA probes can be used. The probes are typically labeled for detecting the corresponding gene (for example, with ³²P, ³H, ³⁵S, biotin, or avidin). Such probes are encompassed by the present invention.

Thus, a genomic DNA or cDNA library prepared from such other organisms may be screened for DNA which hybridizes with the probes described above and which encodes a polypeptide having glucoamylase activity. Genomic or other DNA from such other organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA which is homologous with SEQ ID NO:1 or a subsequence thereof, the carrier material is used in a Southern blot. For purposes of the present invention, hybridization indicates that the nucleic acid sequence hybridizes to a labeled nucleic acid probe corresponding to the nucleic acid sequence shown in SEQ ID NO:1, its complementary strand, or a subsequence thereof, under very low to very high stringency conditions. Molecules to which the nucleic acid probe hybridizes under these conditions are detected using X-ray film.

In a preferred embodiment, the nucleic acid probe is a nucleic acid sequence which encodes the polypeptide of SEQ ID NO:2, or a subsequence thereof. In another preferred embodiment, the nucleic acid probe is SEQ ID NO:1. In another preferred embodiment, the nucleic acid probe is the mature polypeptide coding region of SEQ ID NO:1.

For long probes of at least 100 nucleotides in length, very low to very high stringency conditions are defined as prehybridization and hybridization at 42° C. in 5×SSPE, 0.3% SDS, 200 μg/ml sheared and denatured salmon sperm DNA, and either 25% formamide for very low and low stringencies, 35% formamide for medium and medium-high stringencies, or 50% formamide for high and very high stringencies, following standard Southern blotting procedures.

For long probes of at least 100 nucleotides in length, the carrier material Is finally washed three times each for 15 minutes using 2×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

For short probes which are about 15 nucleotides to about 70 nucleotides in length, stringency conditions are defined as prehybridization, hybridization, and washing post-hybridization at about 5° C. to about 10° C. below the calculated Tₘ using the calculation according to Bolton and McCarthy (1962, Proceedings of the National Academy of Sciences USA 48:1390) in 0.9 M NaCl, 0.09 M Tris-HCl pH 7.6, 6 mM EDTA, 0.5% NP40, 1× Denhardt's solution, 1 mM sodium pyrophosphate, 1 mM sodium monobasic phosphate, 0.1 mM ATP, and 0.2 mg of yeast RNA per ml following standard Southern blotting procedures.

For short probes which are about 15 nucleotides to about 70 nucleotides in length, the carrier material is washed once in 6×SCC plus 0.1% SDS for 15 minutes and twice each for 15 minutes using 6×SSC at 5° C. to 10° C. below the calculated Tₘ. In a preferred embodiment, the polypeptide having glucoamylase activity of the invention comprises a starch-binding-domain (SBD), and preferably the starch-binding-domain comprises an amino acid sequence which is at least 80% identical to the sequence shown in positions 483 to 579 (both incl.) of SEQ ID NO: 2.

Another preferred embodiment relates to the polypeptide of the invention, wherein said polypeptide comprises a linker between the starch-binding domain and the remaining polypeptide of at least 2 amino acids. It may also be preferred, that the polypeptide comprises a signal peptide, for instance it may be preferred that the signal peptide comprises an amino acid sequence which is at least 95% identical to the sequence shown in positions 1 to 18 (both incl.) of SEQ ID NO: 2.

In a preferred embodiment, the present invention relates to variants of the polypeptide having an amino acid sequence of SEQ ID NO:2 comprising a substitution, deletion, and/or insertion of one or more amino acids.

The amino acid sequences of the variant polypeptides may differ from the amino acid sequence of SEQ ID NO:2 or the mature polypeptide thereof by an insertion or deletion of one or more amino acid residues and/or the substitution of one or more amino acid residues by different amino acid residues. Preferably, amino acid changes are of a minor nature, that is conservative amino acid substitutions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter the specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly as well as these in reverse.

In another embodiment, the present invention relates to isolated polypeptides having immunochemical identity or partial immunochemical identity to the polypeptide having the amino acid sequence of SEQ ID NO:2 or the mature polypeptide thereof. The immunochemical properties are determined by immunological cross-reaction identity tests by the well-known Ouchterlony double immunodiffusion procedure. Specifically, an antiserum containing polyclonal antibodies which are immunoreactive or bind to epitopes of the polypeptide having the amino acid sequence of SEQ ID NO:2 or the mature polypeptide thereof are prepared by immunizing rabbits (or other rodents) according to the procedure described by Harboe and Ingild, In N. H. Axelsen, J. Krøll, and B. Weeks, editors, A Manual of Quantitative Immunoelectrophoresis, Blackwell Scientific Publications, 1973, Chapter 23, or Johnstone and Thorpe, Immunochemistry in Practice, Blackwell Scientific Publications, 1982 (more specifically pages 27-31). A polypeptide having immunochemical identity is a polypeptide which reacts with the antiserum in an identical fashion such as total fusion of precipitates, identical precipitate morphology, and/or identical electrophoretic mobility using a specific immunochemical technique. A further explanation of immunochemical identity is described by Axelsen, Bock, and Krøll, In N. H. Axelsen, J. Krøll, and B. Weeks, editors, A Manual of Quantitative Immunoelectrophoresis, Blackwell Scientific Publications, 1973, Chapters 10-11. A polypeptide having partial immunochemical identity is a polypeptide which reacts with the antiserum in a partially identical fashion such as partial fusion of precipitates, partially identical precipitate morphology, and/or partially identical electrophoretic mobility using a specific immunochemical technique.

The antibody may also be a monoclonal antibody. Monoclonal antibodies may be prepared and used, e.g., according to the methods of E. Harlow and D. Lane, editors, 1988, Antibodies, A Laboratory Manual, Cold Spring Harbor Press, Cold Spring Harbor, N.Y.

The polypeptides of the present invention have at least 20%, preferably at least 40%, more preferably at least 60%, even more preferably at least 80%, even more preferably at least 90%, and most preferably at least 100% of the glucoamylase activity of the mature polypeptide of SEQ ID NO:2.

A polypeptide of the present invention may be obtained from microorganisms of any genus. For purposes of the present invention, the term “obtained from” as used herein in connection with a given source shall mean that the polypeptide encoded by the nucleic acid sequence is produced by the source or by a cell in which the nucleic acid sequence from the source has been inserted. In a preferred embodiment, the polypeptide is secreted extracellularly.

A polypeptide of the present invention may be a bacterial polypeptide. For example, the polypeptide may be a gram positive bacterial polypeptide such as a Bacillus polypeptide, e.g., a Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, or Bacillus thuringiensis polypeptide; or a Streptomyces polypeptide, e.g., a Streptomyces lividans or Streptomyces murinus polypeptide; or a gram negative bacterial polypeptide, e.g., an E. coli or a Pseudomonas sp. polypeptide.

A polypeptide of the present invention may be a fungal polypeptide, and more preferably a yeast polypeptide such as a Candida, Kluyveromyces, Pichia, Saccharomyces, Schizosaccharomyces, or Yarrowia polypeptide; or more preferably a filamentous fungal polypeptide such as an Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, or Trichoderma polypeptide.

In a preferred embodiment, the polypeptide is a Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis polypeptide.

In another preferred embodiment, the polypeptide is an Aspergillus aculeatus, Aspergillus awamori, Aspergillus foetidus, Aspergillus japonicus, Aspergillus nidulans, Aspergillus niger, Aspergillus oryzae, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Trichoderma harzianum, Trichoderma koningii, Trichoderma longibrachiatum, Trichoderma reesei, or Trichoderma viride polypeptide.

In another preferred embodiment, the polypeptide is a Thielavia achromatica, Thielavia albomyces, Thielavia albopilosa, Thielavia appendiculata, Thielavia arenaria, Thielavia australiensis, Thielavia basicola, Thielavia californica, Thielavia fimeti, Thielavia fragilis, Thielavia heterothallica, Thielavia hyrcaniae, Thielavia kirilenkoae, Thielavia kiwaitensis, Thielavia leptoderma, Thielavia microspora, Thielavia minuta, Thielavia octospora, Thielavia ovispora, Thielavia peruviana, Thielavia sepedonium, Thielavia setosa, Thielavia spirotricha, Thielavia subthermophila, Thielavia tanzanica, Thielavia terrestris, Thielavia terricola, Thielavia tetraspora, Thielavia thermophila, Thielavia tortuosa, Thielavia variospora, or Thielavia wareingii polypeptide.

In a more preferred embodiment, the polypeptide is a Athelia rolfsii polypeptide, e.g., the polypeptide with the amino acid sequence of SEQ ID NO:2.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents. For example, taxonomic equivalents of Thielavia are defined by Morgan-Jones, 1974, Canadian Journal of Botany 52: 429-431; and Glenn et al., 1996, Mycologia 88: 369-38. For instance, the imperfect form of Thielavia terrestris is known as Acremonium alabamense.

Strains of these species are readily accessible to the public in a number of culture collections, such as the American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

Furthermore, such polypeptides may be identified and obtained from other sources including microorganisms isolated from nature (e.g., soil, composts, water, etc.) using the above-mentioned probes. Techniques for isolating microorganisms from natural habitats are well known in the art. The nucleic acid sequence may then be derived by similarly screening a genomic or cDNA library of another microorganism. Once a nucleic acid sequence encoding a polypeptide has been detected with the probe(s), the sequence may be isolated or cloned by utilizing techniques which are known to those of ordinary skill in the art (see, e.g., Sambrook et al., 1989, supra).

As defined herein, an “isolated” polypeptide is a polypeptide which is essentially free of other non-glucoamylase polypeptides, e.g., at least about 20% pure, preferably at least about 40% pure, more preferably about 60% pure, even more preferably about 80% pure, most preferably about 90% pure, and even most preferably about 95% pure, as determined by SDS-PAGE.

Polypeptides encoded by nucleic acid sequences of the present invention also include fused polypeptides or cleavable fusion polypeptides in which another polypeptide is fused at the N-terminus or the C-terminus of the polypeptide or fragment thereof. A fused polypeptide is produced by fusing a nucleic acid sequence (or a portion thereof) encoding another polypeptide to a nucleic acid sequence (or a portion thereof) of the present invention. Techniques for producing fusion polypeptides are known in the art, and include ligating the coding sequences encoding the polypeptides so that they are in frame and that expression of the fused polypeptide is under control of the same promoter(s) and terminator.

Nucleic Acid Sequences

The present invention also relates to isolated polynucleotides or nucleotide sequences which encode a polypeptide of the present invention. In a preferred embodiment, the nucleic acid sequence is set forth in SEQ ID NO:1. In another preferred embodiment, the nucleic acid sequence is the mature polypeptide coding region of SEQ ID NO:1. The present invention also encompasses nucleic acid sequences which encode a polypeptide having the amino acid sequence of SEQ ID NO:2 or the mature polypeptide thereof, which differ from SEQ ID NO:1 by virtue of the degeneracy of the genetic code. The present invention also relates to subsequences of SEQ ID NO:1 which encode fragments of SEQ ID NO:2 that have glucoamylase activity.

A subsequence of SEQ ID NO:1 is a nucleic acid sequence encompassed by SEQ ID NO:1 except that one or more nucleotides from the 5′ and/or 3′ end have been deleted. Preferably, a subsequence contains at least 1650 nucleotides, more preferably at least 1680 nucleotides, and most preferably at least 1737 nucleotides.

The present invention also relates to mutant nucleic acid sequences comprising at least one mutation in the mature polypeptide coding sequence of SEQ ID NO:1, in which the mutant nucleic acid sequence encodes a polypeptide which consists of amino acids 19 to 471 of SEQ ID NO:2.

The techniques used to isolate or clone a nucleic acid sequence encoding a polypeptide are known in the art and include isolation from genomic DNA, preparation from cDNA, or a combination thereof. The cloning of the nucleic acid sequences of the present invention from such genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleic acid sequence-based amplification (NASBA) may be used. The nucleic acid sequence may be cloned from a strain of Thielavia, Athelia, or another or related organism and thus, for example, may be an allelic or species variant of the polypeptide encoding region of the nucleic acid sequence.

The term “isolated nucleic acid sequence” as used herein refers to a nucleic acid sequence which is essentially free of other nucleic acid sequences, e.g., at least about 20% pure, preferably at least about 40% pure, more preferably at least about 60% pure, even more preferably at least about 80% pure, and most preferably at least about 90% pure as determined by agarose electrophoresis. For example, an isolated nucleic acid sequence can be obtained by standard cloning procedures used in genetic engineering to relocate the nucleic acid sequence from its natural location to a different site where it will be reproduced. The cloning procedures may involve excision and isolation of a desired nucleic acid fragment comprising the nucleic acid sequence encoding the polypeptide, insertion of the fragment into a vector molecule, and incorporation of the recombinant vector into a host cell where multiple copies or clones of the nucleic acid sequence will be replicated. The nucleic acid sequence may be of genomic, cDNA, RNA, semisynthetic, synthetic origin, or any combinations thereof.

The present invention also relates to nucleic acid sequences which have a degree of identity to the mature polypeptide coding sequence of SEQ ID NO:1 of at least about 75%, preferably about 80%, preferably about 85%, more preferably about 90%, even more preferably about 95%, and most preferably about 97% identity, and which encode an active glucoamylase polypeptide. For the purposes of the present invention, the degree of homology between two nucleic acid sequences is determined by the Wilbur-Lipman method (Wilbur and Lipman, 1983, Proceedings of the National Academy of Science USA 80: 726-730) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters were Ktuple=3, gap penalty=3, and windows=20.

Modification of a nucleic acid sequence encoding a polypeptide of the present invention may be necessary for the synthesis of polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variant sequence may be constructed on the basis of the nucleic acid sequence presented as the polypeptide encoding part of SEQ ID NO:1, e.g., a subsequence thereof, and/or by introduction of nucleotide substitutions which do not give rise to another amino acid sequence of the polypeptide encoded by the nucleic acid sequence, but which correspond to the codon usage of the host organism intended for production of the enzyme, or by introduction of nucleotide substitutions which may give rise to a different amino add sequence. For a general description of nucleotide substitution, see, e.g., Ford et al., 1991, Protein Expression and Purification 2: 95-107.

It will be apparent to those skilled in the art that such substitutions can be made outside the regions critical to the function of the molecule and still result in an active polypeptide. Amino acid residues essential to the activity of the polypeptide encoded by the isolated nucleic acid sequence of the invention, and therefore preferably not subject to substitution, may be identified according to procedures known in the art, such as site-directed mutagenesis or alanine-scanning mutagenesis (see, e.g., Cunningham and Wells, 1989, Science 244: 1081-1085). In the latter technique, mutations are introduced at every positively charged residue in the molecule, and the resultant mutant molecules are tested for glucoamylase activity to identify amino acid residues that are critical to the activity of the molecule. Sites of substrate-enzyme interaction can also be determined by analysis of the three-dimensional structure as determined by such techniques as nuclear magnetic resonance analysis, crystallography or photoaffinity labelling (see, e.g., de Vos et al., 1992, Science 255: 306-312; Smith et al., 1992, Journal of Molecular Biology 224: 899-904; Wlodaver et al., 1992, FEBS Letters 309: 59-64).

The present invention also relates to isolated nucleic acid sequences encoding a polypeptide of the present invention, which hybridize under very low stringency conditions, preferably low stringency conditions, more preferably medium stringency conditions, more preferably medium-high stringency conditions, even more preferably high stringency conditions, and most preferably very high stringency conditions with a nucleic acid probe which hybridizes under the same conditions with the nucleic acid sequence of SEQ ID NO:1 or its complementary strand; or allelic variants and subsequences thereof (Sambrook et al., 1989, supra), as defined herein.

Methods for Producing Mutant Nucleic Acid Sequences

The present invention further relates to methods for producing a mutant nucleic acid sequence, comprising introducing at least one mutation into the mature polypeptide coding sequence of SEQ ID NO:1 or a subsequence thereof, wherein the mutant nucleic acid sequence encodes a polypeptide which consists of amino acids 19 to 471 of SEQ ID NO:2 or a fragment thereof which has glucoamylase activity.

The introduction of a mutation into the nucleic acid sequence to exchange one nucleotide for another nucleotide may be accomplished by site-directed mutagenesis using any of the methods known in the art. Particularly useful is the procedure which utilizes a supercoiled, double stranded DNA vector with an insert of interest and two synthetic primers containing the desired mutation. The oligonucleotide primers, each complementary to opposite strands of the vector, extend during temperature cycling by means of Pfu DNA polymerase. On incorporation of the primers, a mutated plasmid containing staggered nicks is generated. Following temperature cycling, the product is treated with Dpnl which is specific for methylated and hemimethylated DNA to digest the parental DNA template and to select for mutation-containing synthesized DNA. Other procedures known in the art may also be used.

Nucleic Acid Constructs

The present invention also relates to nucleic acid constructs comprising a nucleic acid sequence of the present invention operably linked to one or more control sequences which direct the expression of the coding sequence in a suitable host cell under conditions compatible with the control sequences. Expression will be understood to include any step involved in the production of the polypeptide including, but not limited to, transcription, post-transcriptional modification, translation, post-translational modification, and secretion.

“Nucleic acid construct” is defined herein as a nucleic acid molecule, either single- or double-stranded, which is isolated from a naturally occurring gene or which has been modified to contain segments of nucleic acid combined and juxtaposed in a manner that would not otherwise exist in nature. The term nucleic acid construct is synonymous with the term expression cassette when the nucleic acid construct contains all the control sequences required for expression of a coding sequence of the present invention. The term “coding sequence” is defined herein as a nucleic acid sequence which directly specifies the amino acid sequence of its protein product. The boundaries of a genomic coding sequence are generally determined by a ribosome binding site (prokaryotes) or by the ATG start codon (eukaryotes) located just upstream of the open reading frame at the 5′ end of the mRNA and a transcription terminator sequence located just downstream of the open reading frame at the 3′ end of the mRNA. A coding sequence can include, but is not limited to, DNA, cDNA, and recombinant nucleic acid sequences.

An isolated nucleic acid sequence encoding a polypeptide of the present invention may be manipulated in a variety of ways to provide for expression of the polypeptide. Manipulation of the nucleic acid sequence prior to its insertion into a vector may be desirable or necessary depending on the expression vector. The techniques for modifying nucleic acid sequences utilizing recombinant DNA methods are well known in the art.

The term “control sequences” is defined herein to include all components which are necessary or advantageous for the expression of a polypeptide of the present invention. Each control sequence may be native or foreign to the nucleic acid sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide. The term “operably linked” is defined herein as a configuration in which a control sequence is appropriately placed at a position relative to the coding sequence of the DNA sequence such that the control sequence directs the expression of a polypeptide.

The control sequence may be an appropriate promoter sequence, a nucleic acid sequence which is recognized by a host cell for expression of the nucleic acid sequence. The promoter sequence contains transcriptional control sequences which mediate the expression of the polypeptide. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof.

The control sequence may also be a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice may be used in the present invention.

Preferred terminators for filamentous fungal host cells are obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

The control sequence may also be a suitable leader sequence, a nontranslated region of an mRNA which is important for translation by the host cell. The leader sequence is operably linked to the 5′ terminus of the nucleic acid sequence encoding the polypeptide. Any leader sequence that is functional in the host cell of choice may be used in the present invention.

Preferred leaders for filamentous fungal host cells are obtained from the genes for Aspergillus oryzae TAKA amylase and Aspergillus nidulans triose phosphate isomerase.

The control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention.

Preferred polyadenylation sequences for filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

The control sequence may also be a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region which encodes the secreted polypeptide. Alternatively, the 5′ end of the coding sequence may contain a signal peptide coding region which is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used in the present invention.

Effective signal peptide coding regions for filamentous fungal host cells are the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase.

In a preferred embodiment, the signal peptide coding region is nucleotides 1 to 54 of SEQ ID NO:1 which encode amino acids 1 to 18 of SEQ ID NO:2; preferably the signal peptide comprises an amino acid sequence which is at least 95% identical to the sequence shown in positions 1 to 18 (both incl.) of SEQ ID NO: 2.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila laccase (WO 95/33836).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in filamentous fungi are the TAKA alpha-amylase promoter, Aspergillus niger glucoamylase promoter, and Aspergillus oryzae glucoamylase promoter may be used as regulatory sequences. Other examples of regulatory sequences are those which allow for gene amplification. In eukaryotic systems, these include the dihydrofolate reductase gene which is amplified in the presence of methotrexate, and the metallothionein genes which are amplified with heavy metals. In these cases, the nucleic acid sequence encoding the polypeptide would be operably linked with the regulatory sequence.

The present invention also relates to nucleic acid constructs for altering the expression of an endogenous gene encoding a polypeptide of the present invention. The constructs may contain the minimal number of components necessary for altering expression of the endogenous gene. In one embodiment, the nucleic acid constructs preferably contain (a) a targeting sequence, (b) a regulatory sequence, (c) an exon, and (d) a splice-donor site. Upon introduction of the nucleic acid construct into a cell, the construct inserts by homologous. recombination into the cellular genome at the endogenous gene site. The targeting sequence directs the integration of elements (a)-(d) into the endogenous gene such that elements (b)-(d) are operably linked to the endogenous gene. In another embodiment, the nucleic acid constructs contain (a) a targeting sequence, (b) a regulatory sequence, (c) an exon, (d) a splice-donor site, (e) an intron, and (f) a splice-acceptor site, wherein the targeting sequence directs the integration of elements (a)-(f) such that elements (b)-(f) are operably linked to the endogenous gene. However, the constructs may contain additional components such as a selectable marker.

In both embodiments, the introduction of these components results in production of a new transcription unit in which expression of the endogenous gene is altered. In essence, the new transcription unit is a fusion product of the sequences introduced by the targeting constructs and the endogenous gene. In one embodiment in which the endogenous gene is altered, the gene is activated. In this embodiment, homologous recombination is used to replace, disrupt, or disable the regulatory region normally associated with the endogenous gene of a parent cell through the insertion of a regulatory sequence which causes the gene to be expressed at higher levels than evident in the corresponding parent cell. The activated gene can be further amplified by the inclusion of an amplifiable selectable marker gene in the construct using methods well known in the art (see, for example, U.S. Pat. No. 5,641,670). In another embodiment in which the endogenous gene is altered, expression of the gene is reduced.

The targeting sequence can be within the endogenous gene, immediately adjacent to the gene, within an upstream gene, or upstream of and at a distance from the endogenous gene. One or more targeting sequences can be used. For example, a circular plasmid or DNA fragment preferably employs a single targeting sequence, while a linear plasmid or DNA fragment preferably employs two targeting sequences.

The regulatory sequence of the construct can be comprised of one or more promoters, enhancers, scaffold-attachment regions or matrix attachment sites, negative regulatory elements, transcription binding sites, or combinations of these sequences.

The constructs further contain one or more exons of the endogenous gene. An exon is defined as a DNA sequence which is copied into RNA and is present in a mature mRNA molecule such that the exon sequence is in-frame with the coding region of the endogenous gene. The exons can, optionally, contain DNA which encodes one or more amino acids and/or partially encodes an amino acid. Alternatively, the exon contains DNA which corresponds to a 5′ non-encoding region. Where the exogenous exon or exons encode one or more amino acids and/or a portion of an amino acid, the nucleic acid construct is designed such that, upon transcription and splicing, the reading frame is in-frame with the coding region of the endogenous gene so that the appropriate reading frame of the portion of the mRNA derived from the second exon is unchanged.

The splice-donor site of the constructs directs the splicing of one exon to another exon. Typically, the first exon lies 5′ of the second exon, and the splice-donor site overlapping and flanking the first exon on its 3′ side recognizes a splice-acceptor site flanking the second exon on the 5′ side of the second exon. A splice-acceptor site, like a splice-donor site, is a sequence which directs the splicing of one exon to another exon. Acting in conjunction with a splice-donor site, the splicing apparatus uses a splice-acceptor site to effect the removal of an intron.

Expression Vectors

The present invention also relates to filamentous fungal cell recombinant expression vectors comprising a nucleic acid sequence of the present invention, a promoter, and transcriptional and translational stop signals. The various nucleic acid and control sequences described above may be joined together to produce a recombinant expression vector which may include one or more convenient restriction sites to allow for insertion or substitution of the nucleic acid sequence encoding the polypeptide at such sites. Alternatively, the nucleic acid sequence of the present invention may be expressed by inserting the nucleic acid sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) which can be conveniently subjected to recombinant DNA procedures and can bring about the expression of the nucleic acid sequence. The choice of the vector will typically depend on the compatibility of the vector with the host cell into which the vector is to be introduced. The vectors may be linear or closed circular plasmids.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are the amdS and pyrG genes of Aspergillus nidulans or Aspergillus oryzae and the bar gene of Streptomyces hygroscopicus.

The vectors of the present invention preferably contain an element(s) that permits integration of the vector into the host cell's genome or autonomous replication of the vector in the cell independent of the genome.

For integration into the host cell genome, the vector may rely on the nucleic acid sequence encoding the polypeptide or any other element of the vector for integration of the vector into the genome by homologous or nonhomologous recombination. Alternatively, the vector may contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination.

For autonomous replication, the vector may further comprise an origin of replication enabling the vector to replicate autonomously in the host cell in question. The Ama1-sequence is suitable in filamentous fungal host cells.

More than one copy of a nucleic acid sequence of the present invention may be inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

Host Cells

The present invention also relates to recombinant host cells, comprising a nucleic acid sequence encoding a glucoamylase polypeptide of the invention, which are advantageously used in the recombinant production of the polypeptides. A vector comprising a nucleic acid sequence of the present invention is introduced into a host cell so that the vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

In a preferred embodiment, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In another more preferred embodiment, the fungal host cell is a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In an even more preferred embodiment, the filamentous fungal host cell is a cell of a species of, but not limited to, Acremonium, Aspergillus, Fusarium, Humicola, Mucor, Myceliophthora, Neurospora, Penicillium, Thielavia, Tolypocladium, or Trichoderma.

In a most preferred embodiment, the filamentous fungal host cell is an Aspergillus awamori, Aspergillus foetidus, Aspergillus japonicus, Aspergillus nidulans, Aspergillus niger or Aspergillus oryzae cell. In another most preferred embodiment, the filamentous fungal host cell is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In an even most preferred embodiment, the filamentous fungal parent cell is a Fusarium venenatum (Nirenberg sp. nov.) cell. In another most preferred embodiment, the filamentous fungal host cell is a Humicola insolens, Humicola lanuginosa, Mucor miehei, Myceliophthora thermophila, Neurospora crassa, Penicillium purpurogenum, Thielavia terrestris, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus host cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156, and WO 96/00787.

Methods of Production

The present invention also relates to methods of recombinantly producing the glucoamylase, said method comprising the step of expressing a polynucleotide encoding a polypeptide having glucoamylase activity (E.C. 3.2.1.3) in a filamentous fungal host cell, and further comprising (a) cultivating a host cell under conditions conducive for production of the polypeptide; and (b) recovering the polypeptide.

The present invention also relates to methods for producing a polypeptide of the present invention comprising (a) cultivating a host cell under conditions conducive for production of the polypeptide, wherein the host cell comprises a mutant nucleic acid sequence having at least one mutation in the mature polypeptide coding region of SEQ ID NO:1, wherein the mutant nucleic acid sequence encodes a polypeptide which consists of amino acids 19 to 471 of SEQ ID NO:2, and (b) recovering the polypeptide.

The present invention further relates to methods for producing a polypeptide of the present invention comprising (a) cultivating a homologously recombinant cell, having incorporated therein a new transcription unit comprising a regulatory sequence, an exon, and/or a splice donor site operably linked to a second exon of an endogenous nucleic acid sequence encoding the polypeptide, under conditions conducive for production of the polypeptide; and (b) recovering the polypeptide. The methods are based on the use of gene activation technology, for example, as described in U.S. Pat. No. 5,641,670.

In the production methods of the present invention, the cells are cultivated in a nutrient medium suitable for production of the polypeptide using methods known in the art. For example, the cell may be cultivated by shake flask cultivation, and small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptides may be detected using methods known in the art that are specific for the polypeptides. These detection methods may include use of specific antibodies, formation of an enzyme product, or disappearance of an enzyme substrate. For example, an enzyme assay may be used to determine the activity of the polypeptide as described herein.

The resulting polypeptide may be purified or recovered by methods of purification and/or recovery known in the art. For example, the polypeptide may be recovered from the nutrient medium by conventional procedures including, but not limited to, centrifugation, filtration, extraction, spray-drying, evaporation, or precipitation.

The polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989).

Uses

The present invention is also directed to methods for using the polypeptides having glucoamylase activity. The polypeptides may be used in starch conversion processes, especially in the production of dextrose and fructose syrups (U.S. Pat. No. 3,912,590) and in the pre-treatment of starch for extrusion such as for rice and wheat noodles, in production of beer with low carbohydrate content (Manners, The Brewers Digest, December 1974, 56), alcohol from fermentation of raw starch (DE 3638529 C), and in production of wine according to established procedures in the art. The glucoamylase of the invention may also be used in detergents, including laundry detergents, dish wash detergents, and hard surface cleaning compositions. Other uses of the glucoamylase of the invention is in compost and biological waste treatment, in purification of plant extracts for food additives, cosmetics and pharmaceuticals, in the baking industry, including bread and cake production, in a process for producing oligosaccharides, in a process for producing ethanol for fuel or drinking ethanol (portable alcohol), in a process for producing a beverage, in a fermentation process for producing organic compounds, such as citric acid, ascorbic acid, lysine, glutamic acid, in a process for producing specialty syrups, and in the production of pet food.

The glucoamylase variants of the invention may also be used in immobilised form. This is suitable and often used for producing speciality syrups, such as maltose syrups, and further for the raffinate stream of oligosaccharides in connection with the production of fructose syrups.

Ethanol Production

The process of the invention may in one embodiment be an ethanol process comprising the below steps, wherein phytase activity is added during pre-saccharification and/or fermentation. It is to be understood that the glucoamylase according to the invention may be added during the propagation of yeast cells and/or later on during the actual fermentation. Beverage production, such as beer or wine production is equally contemplated.

Alcohol production, in particular ethanol production, from whole grain can be separated into 4 main steps

Milling

Liquefaction

Saccharification

Fermentation

Milling

In one embodiment the (whole) grain is milled in order to open up the structure and allowing for further processing. Two processes are preferred according to the invention: wet and dry milling. Preferred for ethanol production is dry milling where the whole kernel is milled and used in the remaining part of the process. Wet milling may also be used and gives a good separation of germ and meal (starch granules and protein) and is with a few exceptions applied at locations where there is a parallel production of syrups. Both dry and wet milling is well known in the art of, e.g., ethanol production.

Liquefaction

In an embodiment of the liquefaction step of the invention, milled gelatinized whole grain raw material is broken down (hydrolyzed) into maltodextrins (dextrins) mostly of a DE higher than 4. The hydrolysis may be carried out by acid treatment or enzymatically by alpha-amylase treatment, in particular, with a Bacillus alpha-amylase as will be described further below. Acid hydrolysis is used on a limited basis. The raw material is in one embodiment of the invention milled (whole) grain. However, a side stream from starch processing may also be used.

In an embodiment of the invention, enzymatic liquefaction is carried out as a three-step hot slurry process. The slurry is heated to between 60-95° C., preferably 80-85° C., and the enzyme(s) is(are) added to initiate liquefaction (thinning), preferably at least an alpha-amylase is added. Then the slurry is jet-cooked at a temperature between 95-140° C., preferably 105-125° C. to complete gelanitization of the slurry. Then the slurry is cooled to 60-95° C. and more enzyme(s) is (are) added to finalize hydrolysis (secondary liquefaction). The liquefaction process is carried out at pH 4.5-6.5, in particular at a pH between 5 and 6. Milled and liquefied whole grains are known as mash.

The liquefaction step may be performed in the presence of an alpha-amylase derived from a microorganism or a plant. Preferred alpha-amylases are of fungal or bacterial origin. Bacillus alpha-amylases (often referred to as “Termamyl-like alpha-amylases”), variant and hybrids thereof, are specifically contemplated according to the invention. Well-known Termamyl-like alpha-amylases include alpha-amylase derived from a strain of B. licheniformis (commercially available as Termamyl™), B. amyloliquefaciens, and B. stearothermophilus alpha-amylase (BSG). Other Termamyl-like alpha-amylases include alpha-amylase derived from a strain of the Bacillus sp. NCIB 12289, NCIB 12512, NCIB 12513 or DSM 9375, all of which are described in detail in WO 95/26397, and the alpha-amylase described by Tsukamoto et al., Biochemical and Biophysical Research Communications, 151 (1988), pp. 25-31. In the context of the present invention, a Termamyl-like alpha-amylase is an alpha-amylase as defined in WO 99/19467 on page 3, line 18 to page 6, line 27. Contemplated variants and hybrids are described in WO 96/23874, WO 97/41213, and WO 99/19467. Contemplated alpha-amylase derived from a strain of Aspergillus includes Aspergillus oryzae and Aspergillus niger alpha-amylases. Commercial alpha-amylase products and products containing alpha-amylases include TERMAMYL™ SC, FUNGAMYL™, LIQUOZYME™ and SAN™ SUPER.

Fungal alpha-amylases may be added in an amount of 0.001-1.0 AFAU/g DS, preferably from 0.002-0.5 AFAU/g DS, preferably 0.02-0.1 AFAU/g DS. Bacillus alpha-amylases may be added in effective amounts well known to the person skilled in the art.

Saccharification

To produce low molecular sugars DP₁₋₃ that can be metabolized by yeast, the maltodextrin from the liquefaction must be further hydrolyzed. The hydrolysis is typically done enzymatically by glucoamylases, alternatively, alpha-glucosidases or acid alpha-amylases can be used. A full saccharification step may last up to 72 hours, however, it is common only to do a pre-saccharification of typically 40-90 minutes and then complete saccharification during fermentation (SSF). Saccharification is typically carried out at temperatures from 30-65 degrees C., typically around 60 degrees C., and at pH 4.5.

Fermentation

Yeast typically from Saccharomyces spp. is added to the mash and the fermentation is ongoing for 24-96 hours, such as typically 35-60 hours. The temperature is between 26-34 degrees C., in particular about 32□C, and the pH is from pH 3-6, preferably around pH 4-5.

Note that the most widely used process is a simultaneous saccharification and fermentation (SSF) process where there is no holding stage for the saccharification, meaning that fermenting organism, such as the yeast, and enzyme(s) is(are) added together. When doing SSF it is common to introduce a pre-saccharification step at a temperature above 50 degrees C., just prior to the fermentation.

Addition of protease(s) increase(s) the FAN (Free amino nitrogen) level and increase the rate of metabolism of the yeast and further gives higher fermentation efficiency. Suitable proteases include fungal and bacterial proteases. Preferred proteases are acidic proteases, i.e., proteases characterized by the ability to hydrolyze proteins under acidic conditions below pH 7. Suitable acid fungal proteases include fungal proteases derived from Aspergillus, Mucor, Rhizopus, Candida, Coriolus, Endothia, Enthomophtra, Irpex, Penicillium, Sclerotium and Torulopsis. More preferred are proteases derived from Aspergillus niger (see, e.g., Koaze et al., (1964), Agr. Biol. Chem. Japan, 28, 216), Aspergillus saitoi (see, e.g., Yoshida, (1954) J. Agr. Chem. Soc. Japan, 28, 66), Aspergillus awamori (Hayashida et al., (1977) Agric. Biol. Chem., 42(5), 927-933, Aspergillus aculeatus (WO 95/02044), or Aspergillus oryzae; and acidic proteases from Mucor pusillus or Mucor miehei. Bacterial proteases, which are not acidic proteases, include the commercially available products Alcalase® and Neutrase® (available from Novozymes A/S). Protease(s) may in one embodiment be added in an amount of 10⁻⁷ to 10⁻⁵ gram active protease protein/g DS, in particular 10⁻⁷ to 5×10⁻⁵ gram active protease protein/g DS.

Distillation

Optionally following the fermentation, the mash may be distilled to extract, for instance, the ethanol. In the case where the end product is ethanol, obtained according to the process of the invention, it may be used as, e.g., fuel ethanol; drinking ethanol, such as, potable neutral spirits; or industrial ethanol.

Starch Conversion

The present invention provides a method of using glucoamylase of the invention for producing glucose and the like from starch. Generally, the method includes the steps of partially hydrolyzing precursor starch in the presence of α-amylase and then further hydrolyzing the release of D-glucose from the non-reducing ends of the starch or related oligo- and polysaccharide molecules in the presence of glucoamylase by cleaving α-(1,4) and α-(1,6) glucosidic bonds.

The partial hydrolysis of the precursor starch utilizing α-amylase provides an initial breakdown of the starch molecules by hydrolyzing internal α-(1,4)-linkages. In commercial applications, the initial hydrolysis using α-amylase is run at a temperature of approximately 105° C. A very high starch concentration is processed, usually 30% to 40% solids. The initial hydrolysis is usually carried out for five minutes at this elevated temperature. The partially hydrolyzed starch can then be transferred to a second tank and incubated for approximately 1-2 hour at a temperature of 85° to 98° C. to derive a dextrose equivalent (D.E.) of 10 to 15.

The step of further hydrolyzing the release of D-glucose from the non-reducing ends of the starch or related oligo- and polysaccharides molecules in the presence of glucoamylase is normally carried out in a separate tank at a reduced temperature between 30° and 62° C. Preferably the temperature of the substrate liquid is dropped to between 55° and 60° C. The pH of the solution is dropped from about 5.5 to 6.5 to a range between 3 and 5.5. Preferably, the pH of the solution is 4 to 4.5. The glucoamylase is added to the solution and the reaction is carried out for 24-72 hours, preferably 36-48 hours.

By using a thermostable glucoamylase saccharification processes may be carried out at a higher temperature than traditional batch saccharification processes. According to the invention saccharification may be carried out at temperatures in the range from above 60-80° C., preferably 63-75° C. This apply both for traditional batch processes (described above) and for continuous saccharification processes.

Actually, continuous saccharification processes including one or more membrane separation steps, i.e. filtration steps, must be carried out at temperatures of above 60° C. to be able to maintain a reasonably high flux over the membrane or to minimize microbial contamination. Therefore, thermostable glucoamylases provides the possibility of carrying out large scale continuous saccharification processes at a fair price and/or at a lower enzyme protein dosage within a period of time acceptable for industrial saccharification processes. According to the invention the saccharification time may even be shortened.

The glucose yield from a typical saccharification trial with glucoamylase, acid amylase and pullulanase is 95.5-96.5%. The remaining carbohydrates typically consists of 1% maltose, 1.5-2% isomaltose and 1-1.5% higher oligosacharides. The disaccharides are produced since the glucoamylase at high concentrations of glucose and high dry-solid levels has a tendency to form reversion products.

A glucoamylase with an increased specific activity towards saccharides present in the solution after liquefaction and saccharides formed during saccharification would be an advantage as a reduced enzyme protein dosage or a shorter process time then could be used. In general, the glucoamylase has a preference for substrates consisting of longer saccharides compared to short chain saccharides and the specific activity towards e.g. maltoheptaose is therefore approximately 6 times higher than towards maltose. An increased specific activity towards short chain saccharides such as maltose (without reducing the activity towards oligosaccharides) would therefore also permit using a lower enzyme dosage and/or shorter process time.

Furthermore, a higher glucose yield can be obtained with a glucoamylase variant with an increased alpha-1,4 hydrolytic activity (if the alpha-1,6 activity is unchanged or even decreased), since a reduced amount of enzyme protein is being used, and alpha-1,6 reversion product formation therefore is decreased (less isomaltose).

The glucoamylase of the invention may be used in the present inventive process in combination with an enzyme that hydrolyzes only α-(1,6)-glucosidic bonds in molecules with at least four glucosyl residues. Preferentially, the glucoamylase of the invention can be used in combination with pullulanase or isoamylase. The use of isoamylase and pullulanase for debranching, the molecular properties of the enzymes, and the potential use of the enzymes with glucoamylase is set forth in G. M. A van Beynum et al., Starch Conversion Technology, Marcel Dekker, New York, 1985, 101-142.

Signal Peptide

The present invention also relates to nucleic acid constructs comprising a gene encoding a protein operably linked to a nucleic acid sequence consisting of nucleotides 1 to 54 of SEQ ID NO:1 encoding a signal peptide consisting of amino acids 1 to 18 of SEQ ID NO:2, wherein the gene is foreign to the nucleic acid sequence.

The present invention also relates to recombinant expression vectors and recombinant host cells comprising such nucleic acid constructs.

The present invention also relates to methods for producing a protein comprising (a) cultivating such a recombinant host cell under conditions suitable for production of the protein; and (b) recovering the protein.

The nucleic acid sequence may be operably linked to foreign genes with other control sequences. Such other control sequences are described supra.

The protein may be native or heterologous to a host cell. The term “protein” is not meant herein to refer to a specific length of the encoded product and, therefore, encompasses peptides, oligopeptides, and proteins. The term “protein” also encompasses two or more polypeptides combined to form the encoded product. The proteins also include hybrid polypeptides which comprise a combination of partial or complete polypeptide sequences obtained from at least two different proteins wherein one or more may be heterologous or native to the host cell. Proteins further include naturally occurring allelic and engineered variations of the above mentioned proteins and hybrid proteins.

Preferably, the protein is a hormone or variant thereof, enzyme, receptor or portion thereof, antibody or portion thereof, or reporter. In a more preferred embodiment, the protein is an oxidoreductase, transferase, hydrolase, lyase, isomerase, or ligase. In an even more preferred embodiment, the protein is an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase or xylanase.

The gene may be obtained from any prokaryotic, eukaryotic, or other source.

The present invention is further described by the following examples which should not be construed as limiting the scope of the invention.

### EXAMPLES

### Example 1

Cloning of Genomic DNA of Athelia rolfsii into an Aspergillus vector.

The AMG encoding gene was cloned from the genomic DNA of an Athelia rolfsii strain using specific PCR primers that were designed from the AMG sequence which is available in the public databases (e.g. EMBL accession number D49448). The genomic clone contained nine introns which are not present or indicated in the EMBL sequence; the entire sequence encoding the AMG of Athelia rolfsii is shown in SEQ ID NO: 1, the encoded AMG amino acid sequence is shown in SEQ ID NO: 2.

| | |
|---|---|
| Forward cloning primer incl. a | |
| BamH1-site; DCrF1: | |
| 5′ acgtacggatccacaatgtttcgttcactcctgg | (SEQ ID NO:3) |
| Reverse cloning primer incl. a | |
| Sal1-site; DCrR1: | |
| 5′ gtacgtgtcgacctagagaaacaagatagg | (SEQ ID NO:4) |

A PCR amplification with genomic DNA of Athelia rolfsii as template was made with the cloning primers DCrF1 and DCrR1, and the Hi-Fidelity ReddyMix™ (Extensor Hi-Fidelity PCR Master mix ReddyMix™, cat.no AB0794, Abgene®, UK). The PCR product was tested in a 1% agarose gel, and the PCR fragment band at 1876 bp was cut out from the gel and purified using a GFX™ PCR DNA and gel b and purification kit from Amersham Pharmacia (Cat.no 27-9602-01). The PCR fragment was then digested with the two restriction enzymes Sal1 and BamH1 in 2 separate reactions. The fragment was purified using Amersham MicroSpin™ chromatography columns in between the digests.

The Aspergillus expression vector pDAu71 described below was cut with Xho1 and BamH1 at the same time, and was then isolated from a 1% agarose gel. The band at 7857 bp was cut out and purified using the Amersham GFX™ kit.

Purified vector and PCR fragment were ligated with T4 DNA ligase and transformed into E. coli XL1 blue cells. Plasmid DNA was isolated from the resulting clones and was sequenced to confirm correct construction.

Construction of the expression plasmid pMT 2188 was done as follows. The Aspergillus expression plasmid pCaHj527 (described in WO 0070064) consists of an expression cassette based on the Aspergillus niger neutral amylase II promoter fused to the Aspergillus nidulans triose phosphate isomerase non translated leader sequence (Pna2/tpi) and the Aspergillus niger amyloglycosidase terminater (Tamg). Also present on the plasmid is the Aspergillus selective marker amdS from Aspergillus nidulans enabling growth on acetamide as sole nitrogen source and the URA3 marker from Saccharomyces cerevisiae enabling growth of the pyrF defective Escherichia coli strain DB6507 (ATCC 35673). Transformation into E. coli DB6507 using the S. cerevisiae URA 3 gene as selective marker was done in the following way: E. coli DB6507 was made competent by the method of Mandel and Higa (Mandel, M. and A. Higa (1970) J. Mol. Biol. 45, 154). Transformants were selected on solid M9 medium (Sambrook et. al (1989) Molecular cloning, a laboratory manual, 2. edition, Cold Spring Harbor Laboratory Press) supplemented with 1 g/l casaminoacids, 500 μg/l thiamine and 10 mg/l kanamycin. The Pna2/tpi promoter present on pCaHj527 was subjected to site directed mutagenises by a simple PCR approach; the nucleotides 134-144 were altered from GTACTAAAACC to CCGTTAAATTT using a mutagenic primer, and nucleotide 423-436 were altered from ATGCAATTTAAACT to CGGCAATTTAACGG using another mutagenic primer. The resulting plasmid was termed pMT 2188.

Construction of the plasmid pJaL719 took place as follows. The 6352 bp EcoRI-BamHI fragment from pMT2188 and the 617 bp EcoRI-BamHI fragment from pJaL676 where ligated together resulting in plasmid pJaL719.

The plasmid pJaL721 was constructed as follows. Plasmid pJaL721 is a derivate of pJaL719 where part of the NA2 promoter from position 7 to 510 has been duplicated. By PCR a 538 bp DNA fragment was amplified from pJaL676, purified, and digested with the restriction endonucleases EcoRI, resulting in a 520 bp DNA fragment. The 520 bp DNA fragment was ligated together with the 6355 bp EcoRI DNA fragment from pJaL719, resulting in plasmid pJaL721.

The plasmid pCaHj607 was constructed as follows. The ampicillin resistance gene from pUC19 was inserted into the URA3 gene in pMT2188 in such a way that the ampicillin resistance gene was flanked by Not I sites, and so that the URA3 gene could be recreated by excising the ampicillin resistance gene using Not I. The ampicillin resistance gene was PCR amplified from pUC 19 using the PCR primers Amp 5′ and Amp 3′. The 5′ part of the URA3 gene was amplified from pMT2188 using the primers URA amp 5′ and URA 53′ not. The 3′ part of the URA3 gene was amplified from pMT2188 using the primers URA 35′ not and URA amp 3′:

| | |
|---|---|
| amp 3′ (SEQ ID NO:5): | caaagagacatgggcggccgcagg |
| | atcttcacctagatcc |
| Amp 5′ (SEQ ID NO:6): | cgtaaccttcatcgcggccgcatg |
| | tatccgctcatgagac |
| Ura 3 5′ not (SEQ ID NO:7): | gtctcatgagcggatacatgcggc |
| | cgcgatgaaggttacg |
| URA 5 3′ not (SEQ ID NO:8): | ggatctaggtgaagatcctgcggc |
| | cgcccatgtctctttg |
| URA amp 3′ (SEQ ID NO:9): | taatcggtaagcgagttgc |
| URA amp 5′ (SEQ ID NO:10): | ttacgaatgcacacggtgt |

The formed PCR fragments were fuse using the splicing by overlap method again using the primers URA amp 5′ and URA amp 3′ for the amplification. The formed PCR fragment was cloned into pMT2188 as a Stul-EcoRI fragment resulting in the plasmid pCaHj607.

Construction of the Aspergillus expression vector pDAu71 was done as follows. The Aspergillus expression vector pJaL721 was cut with Kspl and Sspl restriction enzymes. The digest was run on 1% agarose gel and the 6558 bp vector band was cut and purified from the agarose gel using the DNA extraction Jetsorb™ gel kit (Genomed®, Germany). A PCR with pJaL721 as template was made using primers DAuP105 and DAuP106, introducing a mutation in the Sspl site.

| | |
|---|---|
| DAuP105 (SEQ ID NO:11): | 5′ gctggtgattggctggct |
| DAuP106 (SEQ ID NO:12): | 5′ atgttgaatagctcgccc |

The Expand™ High Fidelity PCR system (Roche®, Germany) was used according to the manufacturer's instructions. The amplified fragment was then purified from the primers and nucleotides using QIAquick™ DNA cleanup system (Quiagen®). The PCR product was then digested with Kspl restriction enzyme and run on a 2% agarose gel. A 317 bp band was cut and purified from the agarose gel also using the DNA extraction Jetsorb™ kit. The 317 bp fragment was then cloned into the Kspl-Sspl cut vector pJaL721 resulting in the mutagenesis of the Sspl site so that the recognition site did no longer exist. This intermediate vector was named pDAu58.

A stuffer DNA sequence was then cloned into pDAu58 by cutting pDAu58 with BamHI and Xhol restriction enzymes, running the digest on a 1% agarose gel and purifying the cut vector using the Jetsorb™ DNA extraction kit. A stuffer DNA fragment, also cut using BamHI and Xhol, was then cloned into pDAu58. This intermediate vector was named pDAu68.

From plasmid pCaHJ607 an antibiotic selection cassette consisting of the ampicillin resistance gene inserted in the URA3 coding region was then cloned into pDAu68 instead of the URA3 selection marker and the promoter region NA2 minus TATA-box. The antibiotic selection cassette was cut from pCaHJ607 using Xcml and EcoRI restriction enzymes. The Xcml-EcoRI fragment was separated on 1% agarose gel and purified using DNA extraction kit Jetsorb™. The plasmid pDAu68 was also cut with Xcml and EcoRI and purified from a 1% agarose gel using the Jetsorb™ DNA extraction kit. The resulting intermediate plasmid was named pDAu70.

The promoter region NA2 minus TATA-box was then cloned back into pDAu70. The plasmid pJaL721 was cut with EcoRI, the promoter region NA2 minus TATA-box was separated on 1% agarose gel and purified using the DNA extraction Jetsorb™ kit. The promoter region was cloned into pDAu68 cut with EcoRI and purified from a 1% agarose gel using the DNA extraction Jetsorb™ kit. The resulting plasmid was denoted pDAu71.

### Example 2

Making a G2 form of A.rolfsii AMG without Linker and Starch-binding Domain (SBD).

A cloning primer which introduces a STOP-codon in the AMG encoding gene after the sequence which encodes the amino acids IALP was designed.

| | |
|---|---|
| Reverse primer incl. a SacII site; | |
| 240303P1: | |
| 5′ gggcccccgcggctaggggagagcgatcgtggc | (SEQ ID NO:13) |
| actc | |

A PCR amplification with cDNA (ie. intron-free) of Athelia rolfsii as template was made with the cloning primers DCrF1 (SEQ ID NO: 3) and the reverse primer 240303P1 (SEQ ID NO: 13), and the ProofStart™ polymerase from Qiagen®. The PCR product was tested in a 1% agarose gel, and the PCR fragment band was cut out from the gel and purified using a GFX™ PCR DNA and gel band purification kit from Amersham Pharmacia (Cat.no 27-9602-01). The PCR fragment was then digested with the two restriction enzymes SacII and BamH1.

The Aspergillus expression vector pEN12516 (the construction of which is described in detail in co-pending patent application PA 2003 00169, filed with the Danish patent office on 06 Feb. 2003) was cut with SacII and BamHI at the same time, and was then isolated from a 1% agarose gel. The band at 7857 bp was cut out and purified using the Amersham GFX™ kit.

Purified vector and PCR fragment were ligated with T4 DNA ligase and transformed into E.coli One Shots® TOP10 (Invitrogen™) cells. Plasmid DNA was isolated from the resulting clones and was sequenced to confirm correct construction.

### Example 3

Transformation of Aspergillus niger.

90 ml sucrose media+1 ml 1 M NaNO₃+10 ml of YPD (Sherman et al., (1981), Methods in Yeast Genetics, Cold Spring Harbor Laboratory) were inoculated with spores of A. niger strain HowB112 (described in WO 99/28448 A1) and incubated with shaking for about 24 hours. The mycelium was harvested by filtration through miracloth and washed with 200 ml of 0.6 M MgSO4. The mycelium was suspended in 15 ml of 1.2 M MgSO4, 10 mM NaH2PO4, pH 5.8. The suspension was cooled on ice and 40 mg of Novozy™ 234 (Novozymes™) was added. After 5 min., 1 ml of 12 mg/ml BSA (Sigma™ type H25) was added and incubation with gentle agitation continued for 1.5-2 hours at 37 degrees C. until a large number of protoplasts were visible in a sample inspected under the microscope.

The suspension was then filtered through miracloth filter, the filtrate transferred to a sterile tube and overlayed with 5 ml of 0.6 M sorbitol. Centrifugation was performed for 15 min. at 1000 G and the protoplasts were collected from the top of the MgSO4 cushion. 2 volumes of SC (1.2 M sorbitol, 10 mM CaCl2) were added to the protoplast suspension and the mixture was centrifugated for 5 min. at 1000 G. The protoplast pellet was resuspended in 5 ml of SC and repelleted. This was repeated. Finally, the protoplasts were resuspended in 2 ml of SC.

100 microliters of protoplast suspension was mixed with 5 micrograms of DNA prep of A. rolfsii cloning and 1 microgram of the selectionplasmid pToC90 (described in WO 9117243 A1) The mixture was left at room temperature for about 25 minutes. 0.3 ml of 60% PEG 4000 and 10 mM CaCl2 is added and carefully mixed (twice). The mixture was left at room temperature for 25 min. Finally the protoplasts were spread on minimal plates (Cove, (1966), Biochem. Biophys. Acta 113, 51-56) containing 1.0 M sucrose, pH 7.0, 10 mM acetamide as nitrogen source and 20 mM CsCl to inhibit background growth. After incubation for about 7 days at 37 degrees C., spores were picked, inoculated in YPM and spread for single colonies. The YPM supernatant was tested for AMG activity after 3 days incubation at 34 degrees C. This procedure was repeated twice and spores of a single colony after the third re-isolation were stored as a defined transformant.

### Example 4

Saccharification Experiments with A. rolfsii Glucoamylase

Substrates for saccharification were prepared by dissolving a DE 11 maltodextrin prepared from corn starch liquefied with LIQUOZYME X™ (Novozymes A/S) in Milli-Q™ water, and adjusting the dry solid matter content (DS) to approximately 30%. The saccharification experiments were carried out in sealed 50 ml blue-cap flasks fitted with magnetic stirrers. The pH of the solutions were adjusted at the start of saccharification, with the pH electrode calibrated at the saccharification temperature. The following enzymes were used:

| | |
|---|---|
| A. rolfsii GA | 16.5 AGU/ml |
| DEXTROZYME ™ GA (benchmark) | 357 AGU/g |

Standard Reaction Conditions

| | |
|---|---|
| Substrate concentration | 30.0% w/w (initial) |
| Temperature | 60° C. |
| pH (initial) | 4.3 |
| Enzyme dosage | 0.2 AGU/g DS |

Samples were taken at set intervals and heated in boiling water for 15 minutes to inactivate the enzymes. After cooling, the samples were diluted to about 5% DS and filtered (Sartorius MINISART™ NML 0.2 micron), before being analysed by HPLC. The glucose levels as a % of total soluble carbohydrate are given in table 1 below.

TABLE 1

| sample | 24 hours | 48 hours | 72 hours |
|---|---|---|---|
| A. rolfsi GA | 90.74 | 95.91 | 96.29 |
| DEXTROZYME ™ GA | 90.22 | 94.87 | 95.68 |

These results indicate that the purified glucoamylase from A. rolfsii gives a higher glucose yield than the commercial enzyme product DEXTROZYME™ GA (Novozymes A/S; produced from A. niger) which contains both glucoamylase and acid stable amylase activity.

### Example 5

### Saccharification Experiments with A. rolfsii Glucoamylase with Pullulanase

Substrates for saccharification were prepared as above, and the saccharification reactions were carried out in sealed 50 ml blue-cap flasks fitted with magnetic stirrers. The pH's were adjusted at the start of saccharification, with the pH electrode calibrated at the saccharification temperature. The following enzymes were used:

| | |
|---|---|
| A. rolfsii GA | 1.93 AGU/ml |
| DEXTROZYME ™ GA (benchmark) | 357 AGU/g |
| PROMOZYME ™ D2 (pullulanase) | 1480 NPUN/g |

Standard Reaction Conditions

| | |
|---|---|
| Substrate concentration | 30.0% w/w (initial) |
| Temperature | 60° C. |
| pH (initial) | 4.5 |
| Enzyme dosage | see table 2 |

TABLE 2

| | | Dex GA | Promo D2 | |
|---|---|---|---|---|
| Sample | | AGU/g | NPUN/g | A. rolfsii GA |
| no. | Enzyme | DS | DS | AGU/g DS |
| 1 | Dextrozyme ™ GA | 0.20 | | |
| 2 | A. rolfsi + Promozyme ™ | | 0.3 | 0.15 |
| | D2 | | | |

Samples were taken at set intervals and heated in boiling water for 15 minutes to inactivate the enzymes. After cooling, the samples were diluted to about 5% DS and filtered (Sartorius MINISART™ NML 0.2 micron), before being analysed by HPLC. The glucose levels as a % of total soluble carbohydrate are given in table 3 below.

TABLE 3

| Sample no. | 24 hours | 48 hours | 72 hours |
|---|---|---|---|
| 1 | 93.56 | 95.88 | 95.95 |
| 2 | 89.53 | 96.13 | 96.46 |

These results indicate that when purified glucoamylase from A. rolfsii is combined with pullulanase, a higher glucose yield can be achieved than with the commercial product DEXTROZYME™ GA, even though the glucoamylase concentration (enzyme dosage) of the A. rolfsii preparation is lower than that of DEXTROZYME™ GA.

### Example 6

### Saccharification Experiments with A. rolfsii Glucoamylase with A. niger Alpha Amylase

Substrates for saccharification were prepared as above and the saccharification reactions carried out in sealed 50 ml blue-cap flasks fitted with magnetic stirrers. The pH's were adjusted at the start of saccharification, with the pH electrode calibrated at the saccharification temperature. The following enzymes and activities were used:

| | |
|---|---|
| A. rolfsii GA | 2.05 AGU/ml |
| DEXTROZYME ™ GA (benchmark) | 357 AGU/g |
| A. niger acid alpha amylase | 65.4 FAU(A)/g |

Standard Reaction Conditions

| | |
|---|---|
| Substrate concentration | 30.0% w/w (initial) |
| Temperature | 60° C. |
| pH (initial) | 4.3 |
| Enzyme dosage | see table 4 below |

TABLE 4

| | | Dex GA | A. rolfsii | A. niger |
|---|---|---|---|---|
| Sample | | AGU/g | GA | α-amylase |
| no. | Enzyme | DS | AGU/g DS | FAU(A)/g DS |
| 1 | DEXTROZYME ™ | 0.20 | | |
| | GA | | | |
| 2 | A. rolfsii GA + | | 0.175 | 0.042 |
| | A. niger α-amylase | | | |
| 3 | A. rolfsii GA + | | 0.20 | 0.048 |
| | A. niger α-amylase | | | |

Samples were taken at set intervals and heated in boiling water for 15 minutes to inactivate the enzymes. After cooling, the samples were diluted to about 5% DS and filtered (Sartorius MINISART™ NML 0.2 micron), before being analysed by HPLC. The glucose levels as a % of total soluble carbohydrate are given in the table below:

TABLE 5

| Sample no. | 24 hours | 48 hours | 72 hours |
|---|---|---|---|
| 1 | 93.87 | 96.05 | 96.10 |
| 2 | 88.92 | 96.74 | 96.83 |
| 3 | 92.02 | 96.79 | 96.60 |

These results indicate that when purified glucoamylase from A. rolfsii is combined with A. niger acid stable alpha-amylase in the same glucoamylase/alpha-amylase ratio as is found in the commercial product DEXTROZYME™ GA, a significantly higher glucose yield can be achieved than with DEXTROZYME™ GA.

### Example 7

The determination of the % dextrose (DX) value according to the invention is done by Ion Moderated Partition Chromatography, according to the method developed and published by Scobell et al., Cereal Chemistry 54 (4), July-August 1977, pages 905-917. For this analysis, an Aminex HPX 87C column thermostated at 85° C. is recommended. Using deionised water as the eluent, good separation of monosaccharides such as dextrose (glucose) and fructose can be obtained. Monosaccharides and oligosaccharides (maltose, isomaltose, panose and higher sugars) are determined according to the area normalization procedure without the need for calibration and dry matter determination. The amounts of the different components are expressed as % w/w (g/100 g dry matter) as measured by refractometry.

1-43. (canceled)

44. A filamentous fungal host cell comprising at least one copy of a polynucleotide encoding a polypeptide having glucoamylase activity (E.C. 3.2.1.3), wherein the polypeptide comprises an amino acid sequence which is at least 70% identical to the sequence shown in positions 19 to 471 (both incl.) of SEQ ID NO: 2.

45. A method of recombinantly producing a glucoamylase, comprising of expressing a polynucleotide encoding a polypeptide having glucoamylase activity (E.C. 3.2.1.3) in a filamentous fungal host cell of claim 44.

46. The method of claim 45, wherein the polypeptide also comprises a starch-binding-domain (SBD).

47. The method of claim 46, wherein the starch-binding-domain comprises an amino acid sequence which is at least 80% identical to the sequence shown in positions 483 to 579 (both incl.) of SEQ ID NO: 2.

48. The method of claim 44, wherein the polypeptide comprises a linker between the starch-binding domain and the remaining polypeptide of at least 2 amino acids.

49. The method of claim 44, further comprising recovery and/or purification of the polypeptide.

50. A method for saccharifying liquefied starch, comprising the treatment of the liquefied starch with a polypeptide having glucoamylase activity (E.C. 3.2.1.3), wherein the polypeptide comprises an amino acid sequence which is at least 70% identical to the sequence shown in positions 19 to 471 (both incl.) of SEQ ID NO: 2, whereby a % dextrose (DX) value of at least 96% is achieved at 30% w/w (g/100 g dry matter) substrate concentration at 60° C., the DX value is determined as defined in Example 7 herein.

51. The method of claim 50, wherein the polypeptide also comprises a starch-binding-domain (SBD).

52. The method of claim 50, wherein the starch-binding-domain comprises an amino acid sequence which is at least 80% identical to the sequence shown in positions 483 to 579 (both incl.) of SEQ ID NO: 2.

53. The method of claim 50, wherein the polypeptide comprises an amino acid sequence which is at least 80% identical to the sequence shown in positions 19 to 579 (both incl.) of SEQ ID NO: 2.

54. The method of claim 50, wherein the polypeptide comprises a linker between the starch-binding domain and the remaining polypeptide of at least 2 amino acids.
